(19) United States
(12) Patent Application Publication
(10) Pub. No.: US 2011/0144046 A1
(43) Pub. Date: Jun. 16, 2011

(54) ANTHELMINTIC COMPOSITIONS

(76) Inventor: Charles Robert Hillier, Manukau (NZ)

(21) Appl. No.: 13/059,536

(22) PCT Filed: Aug. 18, 2009
(86) PCT No.: PCT/NZ2009/000171

§ 371 Date: Feb. 17, 2011

(30) Foreign Application Priority Data
Aug. 18, 2008 (NZ) 570591

(51) Int. Cl. A61K 31/7048; A61P 33/10

(52) U.S. Cl. 514/30; 514/53

(57) ABSTRACT

An anthelmintic composition comprising two or more active agents, each agent being selected from a separate anthelmintically active family, the families being a macrocyclic lactone (ML), levamisole or its equivalent and a benzimidazole, in a suitable carrier, which includes at least one effective protective agent, which stabilises the chemical and physical activity of the ML, such as hydroxypropyl starch phosphate.

### FIELD OF THE INVENTION

This invention relates to stable anthelmintic compositions having two or more active ingredients.

### BACKGROUND TO THE INVENTION

Parasites in domestic animals are a serious problem. Helminths are one of the more serious parasites. There are three main helminths of economic impact in farmed animals, cestodes, nematodes and trematodes.

Parasitic agents have been developed which are effective against helminths. These anthelmintic agents are generally effective against one of the categories.

Nematodes (otherwise known as round worms) are the most serious from an economic perspective as well as causing most distress to farmed animals, particularly sheep.

Anthelmintic agents effective against round worms are usually categorised as belonging to three different families:

1. The levamisole drenches,

2. The benzimidazoles (BZs)

3. The macrocyclic lactones.

Resistance is a known problem with use of each of these drenches. As one measure to counter resistance, combination formulations containing a drench from two or even three of the families have been developed.

Combining a macrocyclic lactone (ML) with levamisole (usually in the form of a salt, such as the hydrochloride) has been the subject of considerable research. The difficulties of combining these two drenches lies in the stability of each being incompatible in the environment in which the other is stable. Levamisole is stable in acid conditions usually a pH of about 4 or below. The macrocyclic lactones are unstable in conditions where the pH is acidic.

While products have been marketed in which both the ML and levamisole have been combined and marketed with some success, as have drenches combining in addition oxfendazole or albendazole (each being a BZ), there is still a need for alternative formulations in the market.

Thus this invention has as its object the provision of an anthelmintic composition combining a macrocyclic lactone and levamisole in a stable formulation that will at least provide a suitable alternative to existing drenches.

### SUMMARY OF THE INVENTION

In accordance with this invention there is provided an anthelmintic composition comprising a macrocyclic lactone, levamisole or equivalent agent and a carrier, wherein the macrocyclic lactone is stabilised by an effective protecting agent.

The macrocyclic lactone (ML) can be any of those approved for marketing such as abamectin, ivermectin, doramectin, moxidectin or eprinomectin or any macrocyclic lactone which may be approved for marketing in the future.

Levamisole is present in a form that is stable. Usually levamisole will be provided as levamisole hydrochloride which is then dissolved in water. The pH of the solution in water will normally be about the stable level of about 4 or below. pH adjustment of the formulation to the preferred level of about 4 or below can be undertaken with a suitable pH adjusting agent, such as hydrochloric acid. This adjustment can occur once the levamisole in solution is formed or at any stage in the manufacturing process that may be suitable. Buffering to that level is then usually desirable with a suitable buffering agent.

Other anthelmintic agents have been categorised as lying within the same family as levamisole. These include morantel and pyrantel. This invention covers compositions containing an ML and such equivalent agents instead of levamisole.

Other anthelmintic agents can be included. The most preferred third agent is a BZ, particularly albendazole or oxfendazole. The BZs are usually suspended in an aqueous base or organic solvent as they are effective in such insoluble form and are generally insoluble in most solvents, particularly water.

The invention also includes adding an anthelmintic from the recently reported new class of anthelmintics known as the Amino Acetonitrile Derivative (AAD) derivatives. See Kaminsky et al Nature 2008, [AADS] “A new class of anthelmintics effective against drug resistant nematodes”. One such product approved for marketing is monepantel.

Other active agents such as those active against other helminths eg, cestodes , i.e. tape worms and trematodes, i.e. flukes can also be included. For example praziquantel, a tape worm active agent, can be added, usually suspended in the formulation. Similarly closantel, a fluke active either as an insoluble form or as a soluble salt can also be added.

In accordance with this invention the ML is stabilised by a effective protective agent. “effective protective agent” in accordance with the invention means that in a formulation comprising this effective protective agent the ML maintains a level of chemical activity and retains a level of physical stability normally present in a composition containing an ML as the sole active agent. For example, a stable composition results in which the activity of the ML remains within at least 10 per cent of its initial activity for at least three months.

Stability for commercial products is usually measured by a shelf life. A commercial shelf life of at least 12 months, or preferably 15 months, even more preferably 18 months and above, even up to 36 months, is desired. Compositions of the invention having the preferred formulations set forth in the examples are expected to achieve commercially acceptable activity for an acceptable period of time.

The protective agent in the carrier is preferably a modified starch, more preferably hydroxypropyl starch phosphate (HPSP) or any equivalent materials. Such agent can be solely hydroxypropyl starch phosphate or combined with other agents which have a beneficial effect on the stability of the ML. Some degree of trial and error may be required to establish the preferred amount of HPSP, or the equivalent materials and other agents.

Hydroxypropyl starch phosphate (HPSP) is commercially available. It has been marketed for use in personal care products, usually under the brand name Structure XL by National Starch & Chemical Company.

In the compositions of the invention, levamisole usually in the form of its hydrochloride, will be present in an aqueous solution optionally with a BZ in suspension. The ML is normally present in a separate phase at least during preparation of the formulation, such as being suspended in water or dissolved or partially dissolved in a suitable organic liquid. When suspended in water the particle size of the abamectin is desirably substantially uniform. In a typical embodiment all particles are less than 150 μm. Some milling may be needed to achieve the desired size.

The protective agent is normally added to the premix composition containing the ML which is then added to the levamisole premix.

Further, carrier materials will normally be required in order to provide a composition providing acceptable commercial stability.

The compositions of the invention are normally provided in the form of a drench and thus in a form adapted for oral administration. It is within the scope of the invention however for the compositions to be in any suitable form where the benefit of the protective agent can be utilised. For example, pour-on compositions are included within the scope of the invention with suitable carrier materials being provided to ensure the active agents can effectively pass through the skin of the animal.

The particular amount of the active agent required for a particular treatment will vary, depending upon the species, age and weight of the animal being treated, the particular parasite to be guarded against, or treated, as well as the specific active agent selected for the treatment, the route and the frequency of administration.

In the case of oral drenches, the active agents will be present in the compositions in the amounts that are generally recognised as effective. For example, ML at about 2 g/L, the levamisole as the hydrochloride at about 80 g/L and the BZ at about 45 g/L. With pour-ons, it is usual to increase the amount of actives. For example, the ML will generally be present at about 5 g/L.

With some formulations, it has been found that levamisole hydrochloride has tended to degrade initially to a small extent but then plateaus and remains stable. Where that is the situation, the amount of levamisole hydrochloride can be increased above that normally required for activity (usually about 80 g/L) by about 10 % to allow for such initial degradation.

The carriers used in the compositions of the invention, in addition to the protective agent, can contain other suitable materials which may assist in chemically stabilising the active ingredients and provide a physically stable environment for the composition to remain homogeneous. Generally the composition if formed as a suspension desirably remains as such but it is within the scope of the invention for such suspensions where settling out may have occurred to be reformed at the time of application by vigorous shaking of the suitable container.

The compositions of the invention will normally have as the primary vehicle purified water together, if desired, with one or more organic liquids.

Suitable additional organic liquids include benzyl alcohol and propylene glycol octanoate decanoate (marketed under the brand name Miglyol 840). These additional organic liquids can assist in mixing the formulation and may also have some dissolution activity on the ML. But in the preferred formulation it is believed the ML does not need to dissolve in such organic liquids.

Where an organic liquid is used that is not miscible with water and it is desired that some or all of the ML needs to be dissolved in that organic liquid, then it is within the scope of the invention to use an emulsion stabiliser. Preferred formulations do not however require such further stabiliser or dissolution of the ML.

The carrier can in addition contain buffers to buffer the water phase to the desired level for a stable environment for the levamisole hydrochloride or other agents as may be required. As is usual, together with such buffers, a pH adjusting agent can be added. Preservatives, wetting agents, suspension stabilisers, thickeners and other desired materials can also be included.

In addition to anthelmintically active agents, minerals such as cobalt and selenium, can be included.

Suitable buffers include citric acid and trisodium citrate and other citrate salts, such as trisodium citrate dihydrate.

Suitable preservatives include potassium sorbate and benzyl alcohol.

Suitable stabilising agents include colloidal silicon dioxide.

Suitable thickeners include the use of additional HPSP or other known thickeners such as xanthan gum.

A suitable range of individual components for formulations containing two actives and two minerals is described below in Table 1. These ranges are not to be construed as limiting.

TABLE I

| Ingredient | | | g/L |
|---|---|---|---|
| Function | | Example | Dual Drench |
| ML active | | Abamectin | about 2.0 |
| active | | Levamisole HCl | about 80-90 |
| Protective | | Hydroxypropyl Starch Phosphate | about 2-10 |
| agent | | (HPSP) | |
| Organic | ) | Benzyl Alcohol* | about 10-30 |
| liquids | ) | Propylene Glycol Octanoate | about 10-30 |
| | | Decanoate | |
| Buffers | ) | Citric Acid | as required |
| | ) | Trisodium Citrate dihydrate | as required |

| | | |
|---|---|---|
| Preservative | Potassium Sorbate | about 1 to 2 |
| | Benzyl alcohol* | about 10 to 30 |
| Minerals | Cobalt EDTA | ) as required |
| | Sodium Selenate | ) |
| Thickener | HPSP (additional) | 20-40 |
| pH adjuster | Hydrochloric Acid | qs |
| | Purified Water | to 1 L |

*Benzyl alcohol can act as a suitable preservative but is usually used together with another preservative, in this case potassium sorbate.

The protective agent (preferably HPSP) has been found to be effective as a thickener or stabiliser for the whole composition as well as providing its protective effect for the ML. As a thickener or other stabiliser for the whole composition, it will normally be added in a greater amount than that added for the protective effect.

With triple drench formulations containing a ML, levamisole hydrochloride and a BZ drench, HPSP is again preferred as a protective agent. The presence of other thickening agents or stabilising agents rather than HPSP has been found to be desirable in order to maintain the BZ such as oxfendazole in an effective suspended state in the aqueous phase.

The following Table II identifies suitable ranges of the materials for use for such triple drench products, these ranges are not to be construed as limiting.

TABLE II

| Ingredient | | | g/L |
|---|---|---|---|
| Function | | Example | Triple Drench |
| ML active | | Abamectin | about 2 |
| | | Levamisole HCl | about 80-90 |
| BZ active | | Oxfendazole | about 45 |
| Protective | | HPSP | about 2-10 |
| agent | | | |
| Organic | | Benzyl Alcohol* | about 10-30 |
| liquid | | | |
| Buffers | ) | Citric Acid | as required |
| | ) | Trisodium Citrate dihydrate | as required |

| | | |
|---|---|---|
| Preservative | Potassium Sorbate | about 1 to 2 |
| | Benzyl Alcohol* | about 10 to 30 |
| Thickener | Xanthan gum | about 1 to 10 |
| Wetting | Polyoxyl 40 Stearate | about 5-20 |
| agent | | |
| Suspension | Colloidal Silicon Dioxide | about 5-20 |
| stabiliser | | |
| Minerals | Cobalt EDTA | ) as required |
| | Sodium Selenate | ) |
| pH adjuster | Hydrochloric Acid | qs |
| Vehicle | Purified Water | to 1 L |

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

By “w/w” is meant weight/weight, i.e. “1% w/w” means 1 g in 100 g of the composition. “v/v” means volume per volume, and 1% v/v means 1 ml, in a total of 100 ml of the composition, and w/v means weight per volume and 1% w/v means 1 g in a total of 100 ml of the composition.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The formulations of the invention will normally be prepared in two general steps. The macrocyclic lactone mix will be formed by dispersing the ML and at least part of the protective agent in a suitable vessel, and optionally the preservative which will normally be dissolved in the water. Thorough mixing is typically conducted such as with a Silverson mixer.

The levamisole preferably as the hydrochloride is then dissolved in purified water in a separate vessel. Other water soluble ingredients such as buffers, and minerals will typically be added to the purified water in the levamisole hydrochloride mix before or after or simultaneously with the levamisole hydrochloride. The additional thickener or stabiliser can be added to the levamisole hydrochloride mix at any suitable time with suitable stirring so that a homogenous dispersion is formed. The ML mix and levamisole hydrochloride mix can then be combined and again stirred until a homogenous mix is achieved.

The organic liquid when present such as benzyl alcohol can then be added followed by further thorough stirring. Addition of the organic liquid can occur at other stages such as by addition to the ML mix, usually when a triple drench is being produced.

pH adjustment to a pH of 3.7 to 4.0 can then occur using hydrochloric acid and finally purified water is added to achieve the desired volume.

The BZ if present can be added to the levamisole hydrochloride mix to form a suspension before or after addition of the ML mix.

The novel compositions according to the invention have proven good efficacy against internal parasites, especially in sheep. The novel compositions according to the invention result in a unique formulation which controls parasites resistant to ivermectin like, Cooperia and Haemonchus.

The compositions of the invention may be used for the preparation of a medicament for controlling helminth parasites in host animals, particularly in ruminants such as sheep, cattle, buffalos and goats.

The following compositions and manufacturing processes illustrate the invention but are not to be taken as limiting.

### EXAMPLES

| | g/L | |
|---|---|---|
| Ingredients | Dual Drench | Triple Drench |
| Abamectin | 2.0 | 2.0 |
| Levamisole HCl | 80.0 | 80.0 |
| Oxfendazole | — | 45.3 |
| Hydroxypropyl Starch Phosphate | 34 | 3.0 |
| Benzyl Alcohol | 20.0 | 20.0 |
| Propylene Glycol Octanoate Decanoate | 20.0 | — |
| Citric Acid | 10.0 | 10.0 |
| Trisodium Citrate dihydrate | 9.5 | 8.0 |
| Potassium Sorbate | 1.5 | 1.5 |
| Xanthan Gum | — | 2.3 |
| Polyoxyl 40 Stearate | — | 10.0 |
| Colloidal Silicon Dioxide | — | 8.0 |
| Cobalt EDTA | 39.6 | 39.6 |
| Sodium Selenate | 2.4 | 2.4 |
| Hydrochloric Acid | qs | qs |
| Purified Water | To 1 L | To 1 L |

Method of Manufacture

Dual Drench:
Disperse/HPSP (part 1-3 g/l) and abamectin in purified water, dissolve potassium sorbate in the water in a vessel (1). Stir thoroughly with a Silverson mixer.
Dissolve trisodium citrate dihydrate, sodium selenate, citric acid and cobalt EDTA in purified water in a separate vessel (2).
Add and dissolve levamisole hydrochloride in vessel (2).
Add and disperse HPSP (part 2-31 g/l ) in vessel (2) with thorough stirring with a Silverson mixer.
Blend together propylene glycol octanoate decanoate and benzyl alcohol.
Transfer the abamectin dispersion in vessel (1) into the levamisole HCl solution in vessel (2), stir thoroughly.
Transfer the Benzyl alcohol blend into the abamectin/levamisole mixture and stir thoroughly.
Adjust the pH to approx 4
Make up to volume with purified water and homogenise.
Filter through a 400 μm filter.

Triple Drench:
Prepare a gel with xanthan gum and purified water and leave to hydrate in a vessel (1).
Disperse hydroxypropyl starch phosphate and abamectin in purified water, dissolve potassium sorbate, in the water in a vessel (2) with stirring
Add and disperse benzyl alcohol into premix in vessel (2) with stirring.
Heat purified water to 80° C. in a vessel (3).
Add and dissolve polyoxyl 40 stearate in vessel (3) with stirring.
Add and disperse oxfendazole in vessel (3) with stirring.
In purified water in a vessel (4) dissolve trisodium citrate dihydrate, sodium selenate, citric acid anhydrous and cobalt EDTA.
Add and disperse colloidal silicon dioxide and add and dissolve levamisole HCL with stirring in vessel (4).
Transfer the abamectin dispersion in a vessel (2) into the levamisole solution in a vessel (4) with stirring.
Transfer the oxfendazole dispersion in vessel (3) into the levamisole solution in vessel (4) with stirring.
Adjust the pH to approximately pH 4.
Add the xanthan gum gel in vessel (1) to the bulk in vessel (4) with stirring, top up with purified water and homogenize.

Note

Stirring at each stage is thorough using a Silverson mixer.

Stability trials have been conducted in accordance with those acceptable to regulatory authorities in New Zealand. Trials with the dual drench and triple drench have indicated that a shelf life of over 18 months has been achieved.

Efficacy trials were conducted on cattle and sheep. In the cattle trials, the dual drench product of example 1 was compared to two known pour-on products marketed in New Zealand, one containing eprinomectin and the other containing abamectin and levamisole HCL.

The results indicated that the dual drench of the invention had satisfactory efficacy in comparison to the other two products at four different sites throughout New Zealand.

Sheep trials compared the dual formulation of example 1 with three drenches available in New Zealand, one containing levamisole hydrochloride, a second containing abamectin and a third containing abamectin and levamisole hydrochloride. The trials were conducted on farms having a diagnosed history of ML resistance. The results showed the dual formulation of example 1 was equally effective on all farms as that of the third product and better than the first and second products.

While the invention has been described with reference to preferred embodiments, it is not to be construed as limited thereto. Moreover, where specific materials or operational steps have been described and equivalents are known to exist thereto, such equivalent materials and steps are incorporated herein as if specifically set forth.

### INDUSTRIAL APPLICABILITY

The invention provides a composition to treat helminths in animals to assist in economically farming the animals.

1. An anthelmintic composition comprising a macrocyclic lactone, levamisole or equivalent agent and a carrier, wherein the macrocyclic lactone is stabilised by an effective protecting agent.

2. A composition as claimed in claim 1 in which the macrocyclic lactone is selected from the group consisting of abamectin, ivermectin, doramectin, moxidectin or eprinomectin.

3. A composition as claimed in claim 1 or 2 in which the macrocyclic lactone is suspended at least in part in the carrier which comprises water.

4. A composition as claimed in claim 1 or 2 or 3 in which the levamisole is present as levamisole hydrochloride.

5. A composition as claimed in claim 4 in which the levamisole hydrochloride is dissolved in water.

6. A composition as claimed in claim 5 in which the pH of the solution in water is about 4 or below.

7. A composition as claimed in claim 6 in which the composition is buffered to the desired pH with a suitable buffering agent.

8. A composition as claimed in claim 1 or 2 in which levamisole is replaced with an equivalent agent, such as morantel or pyrantel.

9. A composition as claimed in any one of the preceding claims in which a third active agent selected from a benzimidazole is present.

10. A composition as claimed in claim 9 in which the benzimidazole is albendazole or oxfendazole.

11. A composition as claimed in claim 10 in which the benzimidazole is suspended in the composition.

12. A composition as claimed in any one of the preceding claims containing in addition a further active agent.

13. A composition as claimed in claim 12 in which the further active agent is selected from anthelmintics that are active against other helminths.

14. A composition as claimed in claim 13 in which the composition contains agents effective against cestodes and/or trematodes.

15. A composition as claimed in any one of the preceding claims wherein the protective agent is a modified starch.

16. A composition as claimed in any one of the preceding claims in which the protective agent is hydroxypropyl starch phosphate (HPSP).

17. A composition as claimed in claim 16 in which the hydroxypropyl starch phosphate is present from about 0.2 to about 1.0% w/v.

18. A composition as claimed in claim 16 in which hydroxypropyl starch phosphate is present in an amount sufficient to stabilise the macrocyclic lactone and in addition is present as a thickener or suspension stabiliser for the whole composition.

19. A composition as claimed in claim 18 in which the hydroxypropyl starch phosphate is present as a protective agent in an amount from about 0.2% to about 1.0% w/v, and is present as a thickener or stabiliser for the whole composition in an amount of from about 2.0% to about 4.0% w/v.

20. A composition as claimed in any one of the preceding claims in which the composition is formed by suspending the macrocyclic lactone in an aqueous phase, homogenously mixing with the protective agent, adding that to a preformed mixture of levamisole dissolved in water, optionally adding at any stage of the process one or more other carrier materials and optionally adding a further active agent such as a benzimidazole.

21. A composition as claimed in any one of the preceding claims in a form suitable for oral or pour-on administration.

22. A composition as claimed in any one of the preceding claims containing in addition preservatives, wetting agents, suspension stabilisers, thickeners and/or minerals.

23. A composition substantially as set forth in Table 1 herein.

24. A composition substantially as set forth in Table 2 herein.

25. A composition as claimed in claim 1 in which hydroxypropyl starch phosphate is present as the protective agent and in addition the composition contains other thickeners, wetting agents and suspension stabilisers in order to provide a physically stable environment.

26. A composition substantially as set forth in the example headed ‘Dual Drench”.

27. A composition substantially as set forth in the example headed “Triple Drench”.

28. A process for producing a composition as claimed in claim 1 comprising forming in two parts separate components for the composition, the first part containing the macrocyclic lactone and the protective agent and the second part containing levamisole in a suitable vehicle and then adding the first part to the second part and stirred to provide a stable formulation.

29. A process as claimed in claim 28 in which the components are stirred thoroughly during the formation of each part.

30. A process as claimed in claim 28 or claim 29 in which the macrocyclic lactone and protective agent are suspended in water in the first part.

31. A process as claimed in claim 30 in which the protective agent is added in two separate stages, the first stage being to the first part of the composition to protect the macrocyclic lactone and the second stage being to the second part containing the levamisole to stabilise the whole composition.

32. A process as claimed in claim 31 wherein a thickening agent is substituted for the protective agent in the second stage.

33. A process as claimed in any one of claims 28 to 32 in which an organic liquid, preferably benzyl alcohol is added to the composition.

34. A process as claimed in claim 33 in which the organic liquid is added to the composition after combining the first part and the second part.

35. Use of the composition as claimed in any one of claims 1-27 for the manufacture of a medicament for the control of helminth parasites in animals.
